(19) United States
(12) Patent Application Publication
(10) Pub. No.: US 2022/0160448 A1
(43) Pub. Date: May 26, 2022

(54) SYSTEMS AND METHODS FOR MAINTAINING STERILITY OF A COMPONENT USING A MOVABLE, STERILE VOLUME

(71) Applicant: Intuitive Surgical Operations, Inc., Sunnyvale, CA (US)

(72) Inventors: Daniel H. Gomez, Los Gatos, CA (US); Ryan C. Abbott, San Jose, CA (US); John Ryan Steger, Los Gatos, CA (US)

(21) Appl. No.: 17/440,349

(22) PCT Filed: Mar. 20, 2020
(86) PCT No.: PCT/US2020/023918

§ 371 Date: Sep. 17, 2021

Related U.S. Application Data
(60) Provisional application No. 62/822,350, filed on Mar. 22, 2019.

(51) Int. Cl. A61B 34/00; A61B 46/10; A61B 34/35

(52) U.S. Cl. CPC A61B 34/70; A61B 2090/571; A61B 34/35; A61B 46/10

(57) ABSTRACT

A system includes a manipulator assembly and a shroud that defines a sterile volume. The manipulator assembly includes a sterile link or a non-sterile link covered by an external sterile cover, and the link is received into and withdrawn from the shroud's sterile volume. The outside of the shroud may be non-sterile while the shroud maintains its interior sterile volume, so that the link or external sterile cover remains sterile as it moves within the shroud's sterile volume. The shroud may then extend into a non-sterile field in a surgical environment, and the link or external sterile cover remains sterile as the link is moved from the non-sterile field into a sterile field for surgery. The shroud may be movable, it may mechanically support the manipulator assembly, and it may be coupled to a surgical table or to a unit separate from a surgical table.

### CROSS-REFERENCE TO RELATED APPLICATIONS

This application claims the benefit of U.S. Provisional Application No. 62/822,350, filed Mar. 22, 2019, which is incorporated by reference herein in its entirety.

### FIELD

The present disclosure is directed to a sterile shroud of a teleoperated surgical manipulator system.

### BACKGROUND

Computer-assisted devices often include one or more movable manipulators operable to manipulate instruments for performing a task at a work site. The computer-assisted devices may include at least one movable manipulator for supporting a medical instrument, such as an image capturing device that captures images of the work site or a surgical instrument that may be used to manipulate or treat tissue at the surgical work site. A movable manipulator can include interconnected links that are coupled together by one or more actively controlled joints. The manipulator can include one or more passive joints that are not actively controlled and comply with movement of an actively controlled joint. The active and passive joints can be locked to hold the movable manipulator in place.

The computer-assisted devices can include industrial and recreational systems, and also medical robotic systems used in procedures for diagnosis, cosmetics, therapeutics, non-surgical treatment, surgical treatment, etc. As a specific example, computer-assisted devices include minimally invasive, computer-assisted, teleoperated surgical systems (“telesurgical systems”) that allow a surgeon to operate on a patient from bedside or a remote location. Telesurgery is a general term for surgical systems in which the surgeon, rather than directly holding and moving all parts of the instruments by hand, uses some form of indirect or remote control, e.g., a servomechanism, or the like, to manipulate surgical instrument movements with at least partial computer assistance. The surgical instruments for such surgical systems are inserted through minimally invasive surgical apertures or natural orifices to treat tissues at sites within the patient, often reducing the trauma generally associated with accessing a surgical worksite by open surgery techniques.

During a surgical procedure, a surgical environment, such as an operating room, may have both a sterile field and a non-sterile field. If a sterile object moves from the sterile field into the non-sterile field, the object is then considered non-sterile because there is a risk of contamination if the object is re-introduced into the sterile field. Therefore, it would be advantageous to maintain the sterility of a sterile object that moves from a sterile field into a non-sterile field and then back into the sterile field. More specifically, it would be advantageous to maintain the sterility of a telesurgical system device or device component if it moves out of a sterile surgical field defined for a patient under surgery and then reenters the sterile surgical field so that it does not contaminate the sterile field.

### SUMMARY

Embodiments of the present disclosure are summarized by the claims that follow the description.

Consistent with some embodiments, to maintain sterility in the context of a telesurgical system that is adjacent to, attached to, or an integral part of an operating table, the present disclosure provides a local extension of the typical operating room sterile field into a portion of the non-sterile field within the protected confines of a sterile shroud that is added to the telesurgical system to receive the portion of the telesurgical system that moves in and out of the sterile field.

Consistent with some embodiments, a system is provided. The system includes a shroud that defines a sterile volume. The system further includes a manipulator assembly including a sterile link slidingly received within the sterile volume. The link includes an external sterile surface or is covered by an external sterile cover positioned at least partially between the shroud and the link.

Consistent with other embodiments, a method includes extending a link of a manipulator assembly from a sterile volume defined by a shroud to a sterile field, the shroud being at least partially within a non-sterile field of a surgical environment. The link includes an external sterile surface or is covered by an external sterile cover positioned at least partially between the shroud and the link. Other embodiments include corresponding computer systems, apparatus, and computer programs recorded on one or more computer storage devices, each configured to perform the actions of the methods.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1A is a simplified diagram of a computer-assisted, teleoperated system according to some embodiments.

FIG. 1B is a simplified diagram of a teleoperated surgical manipulator assembly mounted on a support base according to some embodiments.

FIG. 1C is a simplified diagram of a teleoperated surgical manipulator assembly mounted on a support base according to some embodiments.

FIG. 2 is a perspective view of a patient coordinate space including a teleoperated surgical manipulator assembly mounted on a side of a surgical table according to some embodiments.

FIG. 3A is a perspective view of a teleoperated surgical manipulator assembly in a retracted position mounted on a side of a surgical table according to some embodiments.

FIG. 3B is a perspective view of a teleoperated surgical manipulator assembly in an extended position mounted on a side of a surgical table according to some embodiments.

FIG. 4A is a cross-sectional view, along section line 4A-4A in FIG. 3A, of a teleoperated surgical manipulator system in a retracted position mounted on a side of a surgical table according to some embodiments.

FIG. 4B is a cross-sectional view, along section line 4B-4B in FIG. 3B, of a teleoperated surgical manipulator system in an extended position mounted on a side of a surgical table according to some embodiments.

FIG. 5 is a cross-sectional view of a teleoperated surgical manipulator system in an extended position according to some embodiments.

FIG. 6 is a perspective view of a teleoperated surgical manipulator system coupled to a kinematic arm mounted on a side of a surgical table according to some embodiments.

FIG. 7A is a perspective view of a teleoperated surgical manipulator system coupled to a kinematic arm mounted on a movable manipulator system according to some embodiments.

FIG. 7B is a perspective view of a teleoperated surgical manipulator system coupled to a kinematic arm mounted on a movable manipulator system according to some embodiments.

FIG. 8 illustrates a method for extending a sterile link from a non-sterile field to a sterile field according to some embodiments.

Embodiments of the present disclosure and their advantages are best understood by referring to the detailed description that follows. It should be appreciated that like reference numerals are used to identify like elements illustrated in one or more of the figures for purposes of illustrating but not limiting embodiments of the present disclosure.

### DETAILED DESCRIPTION

In the following description, specific details describe some embodiments consistent with the present disclosure. Numerous specific details are set forth in order to provide a thorough understanding of the embodiments. It will be apparent to one skilled in the art, however, that some embodiments may be practiced without some or all of these specific details. The specific embodiments disclosed herein are meant to be illustrative but not limiting. One skilled in the art may realize other elements that, although not specifically described, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances well known methods, procedures, components, and circuits have not been described in detail so as not to unnecessarily obscure aspects of the embodiments.

Further, specific words chosen to describe one or more embodiments and optional elements or features are not intended to limit the present disclosure. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various special device positions and orientations. The combination of a body's position and orientation define the body's pose.

Similarly, geometric terms, such as “parallel” and “perpendicular” are not intended to require absolute mathematical precision, unless the context indicates otherwise. Instead, such geometric terms allow for variations due to manufacturing or equivalent functions.

In addition, the singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. The auxiliary verb “may” likewise implies that a feature, step, operation, element, or component is optional.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system”, are analogous.

Although some of the examples described herein refer to surgical procedures or instruments, or medical procedures and medical instruments, the techniques disclosed optionally apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy), and performing procedures on human or animal cadavers. Further, these techniques can also be used for surgical and nonsurgical medical treatment or diagnosis procedures.

Further, although some of the examples presented in this disclosure discuss teleoperational robotic systems or remotely operable systems, the techniques disclosed are also applicable to computer-assisted systems that are directly and manually moved by operators, in part or in whole.

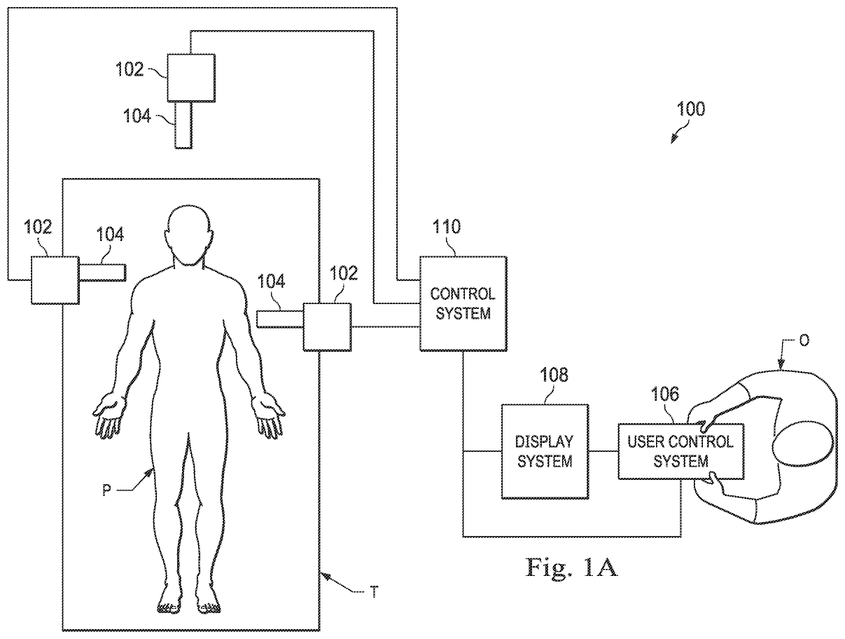

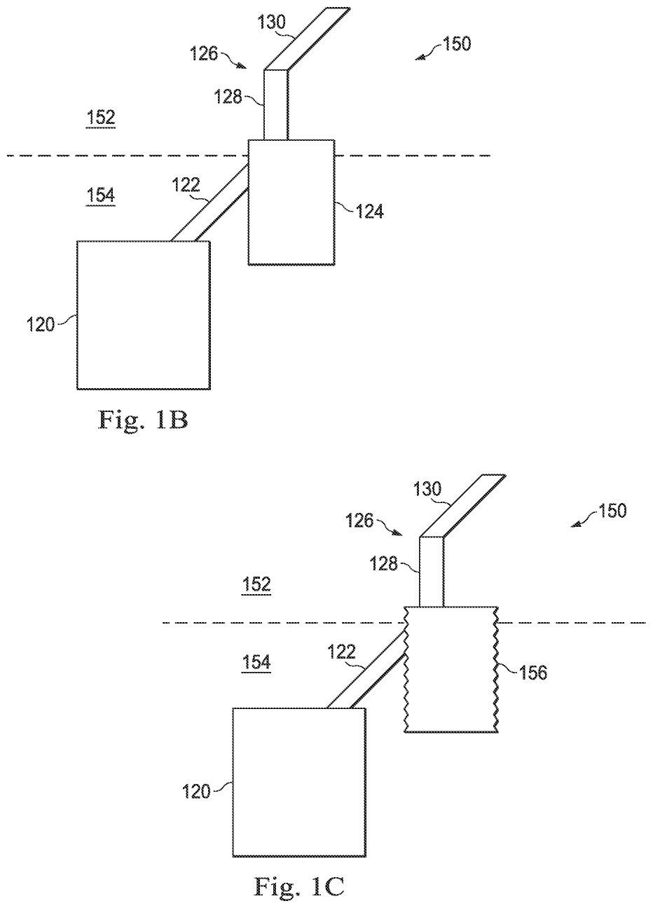

FIG. 1A is a simplified diagram of a computer-assisted, teleoperated system 100 according to some embodiments. In some embodiments, system 100 may be suitable for use in therapeutic and diagnostic procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic, general teleoperational, or robotic medical systems.

As shown in FIG. 1A, system 100 generally includes a plurality of manipulator assemblies 102. Although three manipulator assemblies 102 are illustrated in the embodiment of FIG. 1A, in other embodiments, more or fewer manipulator assemblies may be used. The exact number of manipulator assemblies will depend on the medical procedure and the space constraints within the operating room, among other factors.

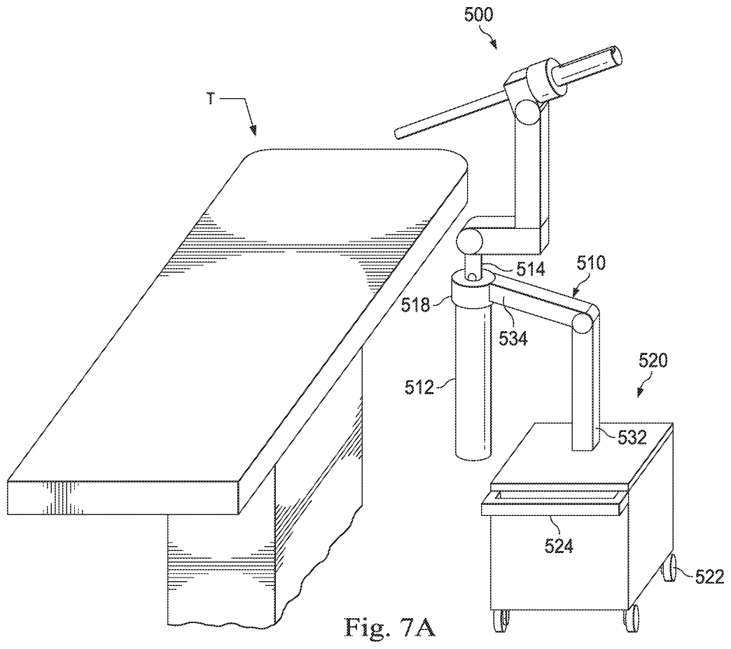

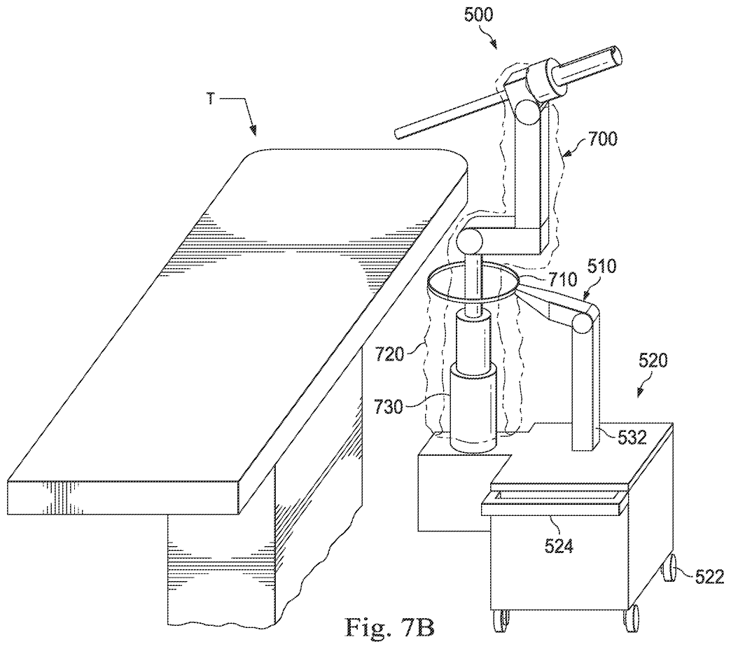

The manipulator assembly 102 is used to operate a medical instrument 104 (e.g., a surgical instrument or an image capturing device) in performing various procedures on a patient P. The manipulator assembly 102 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. In some embodiments, the manipulator assembly 102 may be mounted near or adjacent an operating or surgical table T, or the manipulator assembly 102 may be mounted directly to the table T, or to a rail coupled to the table T, or integrally part of the table structure. In some embodiments, the manipulator assembly 102 may be mounted to a movable cart (e.g., a patient-side cart), as described in more detail with respect to FIGS. 7A-7B below. The movable cart may be separate from and spaced from the table T in the operating room and may be independently movable relative to the table T. In some embodiments, the movable cart may be docked or attached to the table T. The manipulator assembly 102 may be mounted to a ceiling, floor, and/or wall of the operating room. In embodiments in which a plurality of manipulator assemblies 102 are employed, one or more of the manipulator assemblies 102 may support surgical instruments, and another of the manipulator assemblies may support an image capturing device, such as a monoscopic or stereoscopic endoscope. In such embodiments, one or more of the manipulator assemblies 102 may be mounted to any structure or in any manner as described above. For example, one manipulator assembly 102 may be mounted to the table T and another manipulator assembly 102 may be mounted to a manipulator platform.

A user control system 106 allows an operator (e.g., a surgeon or other clinician as illustrated in FIG. 1A) to view the interventional site and to control manipulator assembly 102. In some examples, the user control system 106 is a surgeon console, which is usually located in the same room as the operating or surgical table T, such as at the side of a table on which patient P is located. It is to be understood, however, that operator O can be located in a different room or a completely different building from patient P. That is, one or more user control systems 106 may be collocated with the manipulator assemblies 102, or the user control systems may be positioned in separate locations. Multiple user control systems allow more than one operator to control one or more teleoperated manipulator assemblies in various combinations.

User control system 106 generally includes one or more input devices for controlling manipulator assembly 102. The input devices may include any number of a variety of devices, such as joysticks, trackballs, data gloves, trigger-guns, hand-operated controllers, voice recognition devices, body motion or presence sensors, and/or the like. To provide operator O a strong sense of directly controlling medical instrument 104, the input devices may be provided with the same degrees of freedom as the associated medical instrument 104. In this manner, the input devices provide operator O with telepresence and the perception that the input devices are integral with medical instrument 104.

Manipulator assembly 102 supports medical instrument 104 and may include a kinematic structure of one or more non-servo controlled links (e.g., one or more links that may be manually positioned and locked in place, generally referred to as a set-up structure), and/or one or more servo controlled links (e.g., one or more links that may be controlled in response to commands from a control system), and a manipulator. Manipulator assembly 102 may optionally include a plurality of actuators or motors that drive inputs on medical instrument 104 in response to commands from the control system (e.g., a control system 110). The actuators may optionally include drive systems that when coupled to medical instrument 104 may advance medical instrument 104 into a naturally or surgically created anatomic orifice. Other drive systems may move the distal end of medical instrument 104 in multiple degrees of freedom, which may include three degrees of linear motion (e.g., linear motion along the X, Y, Z Cartesian axes) and three degrees of rotational motion (e.g., rotation about the X, Y, Z Cartesian axes). Additionally, the actuators can be used to actuate an articulable end effector of medical instrument 104 for grasping tissue in the jaws of a biopsy device and/or the like. Actuator position sensors such as resolvers, encoders, potentiometers, and other mechanisms may provide sensor data to system 100 describing the rotation and orientation of the motor shafts. This position sensor data may be used to determine motion of the objects manipulated by the actuators. The manipulator assembly 102 may position its held instrument 104 so that a pivot point occurs at the instrument's entry aperture into the patient. The manipulator assembly 102 may then manipulate its held instrument so that the instrument may be pivoted about the pivot point, inserted into and retracted out of the entry aperture, and rotated about its shaft axis.

System 100 also includes a display system 108 for displaying an image or representation of the surgical site and medical instrument 104. Display system 108 and user control system 106 may be oriented so operator O can control medical instrument 104 and user control system 106 with the perception of telepresence. In some examples, the display system 108 may present images of a surgical site recorded pre-operatively or intra-operatively using image data from imaging technology such as, computed tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

System 100 also includes control system 110. Control system 110 includes at least one memory and at least one computer processor (not shown) for effecting control between medical instrument 104, user control system 106, and display system 108. Control system 110 also includes programmed instructions (e.g., a non-transitory machine-readable medium storing the instructions) to implement some or all of the methods described in accordance with aspects disclosed herein, including instructions for providing information to display system 108. While control system 110 is shown as a single block in the simplified schematic of FIG. 1A, the system may include two or more data processing circuits with one portion of the processing optionally being performed on or adjacent to manipulator assembly 102, another portion of the processing being performed at user control system 106, and/or the like. The processors of control system 110 may execute instructions comprising instruction corresponding to processes disclosed herein and described in more detail below. Any of a wide variety of centralized or distributed data processing architectures may be employed. Similarly, the programmed instructions may be implemented as a number of separate programs or subroutines, or they may be integrated into a number of other aspects of the robotic medical systems described herein. In one embodiment, control system 110 supports wireless communication protocols such as Bluetooth, IrDA, HomeRF, IEEE 802.11, DECT, and Wireless Telemetry.

Movement of a manipulator assembly 102 may be controlled by the control system 110 so that a shaft or intermediate portion of instruments mounted to the manipulator assemblies 102 are constrained to safe motions through minimally invasive surgical access sites or other apertures. Such motion may include, for example, axial insertion of a shaft through an aperture site, rotation of the shaft about its axis, and pivotal motion of the shaft about a pivot point adjacent the access site. In some cases, excessive lateral motion of the shaft that might otherwise tear the tissues adjacent the aperture or enlarge the access site inadvertently is inhibited. Some or all of such constraint on the motions of the manipulator assemblies 102 at the access sites may be imposed using mechanical manipulator joint linkages that inhibit improper motions, or may in part or in full be imposed using data processing and control techniques. In some embodiments, control system 110 may receive force and/or torque feedback from medical instrument 104. Responsive to the feedback, control system 110 may transmit signals to user control system 106. In some examples, control system 110 may transmit signals instructing one or more actuators of manipulator assembly 102 to move medical instrument 104.

FIG. 1B is a simplified diagram of a manipulator assembly 126 mounted on a support base 120 according to some embodiments. In some embodiments, the manipulator assembly 126 may be used as manipulator assembly 102 in a medical procedure. The manipulator assembly 126 may be used for computer-assisted teleoperated surgical procedures or in procedures that also involve traditional manually operated minimally invasive surgical instruments, such as manual laparoscopy.

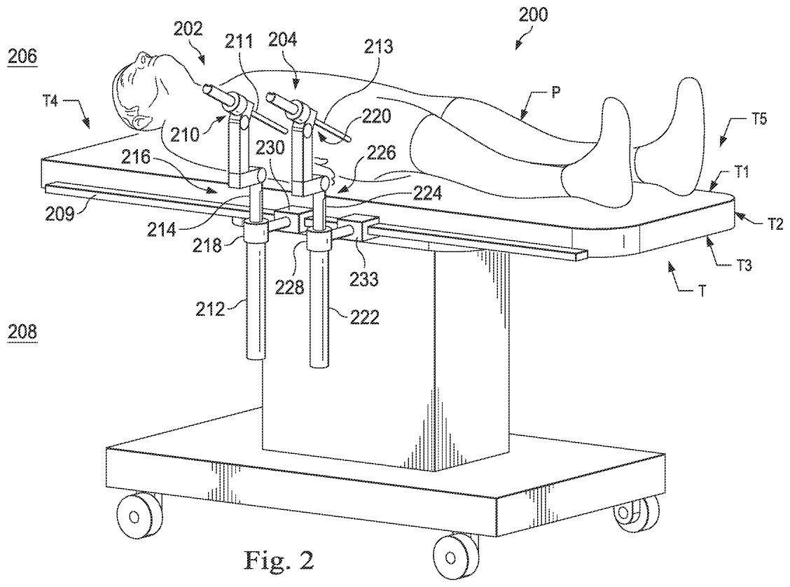

As shown, a patient coordinate space 150 includes a sterile field 152 (e.g., corresponding to the sterile field 206 in FIG. 2) and a non-sterile field 154 (e.g., corresponding to the non-sterile field 208 in FIG. 2). In some embodiments, the sterile field 152 and the non-sterile field may be defined volumes. By way of example, the manipulator assembly 126 is comprised of two components 128, 130, which may be operated to move within the patient coordinate space 150. In some embodiments, the components 128, 130 move an instrument coupled to one of the components. As shown, the component 130 is an example of a teleoperated manipulator, and component 128 is an example of a kinematic support structure (e.g., a serial kinematic arm) that supports the component 130. Both components 128 and 130 optionally include one or more links or kinematic pairs of links, which may be manually positionable and locked into place and/or may include actuators or motors that are driven in response to commands from the control system. The component 128 includes one or more links that support the component 130. During a medical procedure, some or all of the manipulator assembly 126 operates within the sterile field 152, and so some or all of the components 128 and 130 that operate within sterile field 152 are either covered with a sterile drape (to make their external contactable surfaces sterile) and/or sterilized before surgery and must remain sterile during surgery.

The manipulator assembly 126 is coupled to a shroud 124. In some embodiments, the shroud 124 is a rigid member, such as a cylindrical tube, a rectangular prism, a pentagonal prism, a hexagonal prism, or any other suitable elongated and/or concave shape. In alternative embodiments, the shroud 124 is a non-rigid member made of, for example, cloth, paper, plastic, rubber, a treated material, a laminated material, a layered material, or any other suitable flexible material. Some or all of the support structure component 128 may be received within the shroud 124, for example by sliding, folding, or telescoping motion, that allows component 128 to become entirely or partially received within the shroud 124. In some embodiments, some or all of both components 128 and 130 are received within the shroud 124. In addition, the manipulator assembly 126 may include several components as part of or in addition to the components 128, 130 (e.g., an instrument, a handle, additional linkage members, etc.), and some or all of these other components of the manipulator assembly 126 discussed above may also be received within the shroud 124 along with the components 128, 130. In alternative embodiments, any one or more of the components of the manipulator assembly 126 discussed above may be received within the shroud 124, alone or in combination with the remaining components of the manipulator assembly 126.

In some embodiments, the portion of the components of the manipulator assembly 126 received within the shroud 124 may be non-sterile. In such embodiments, any one or more of the components being received within the shroud 124, may themselves have an external sterile cover such as a sterile drape, sterile sleeve, or sterile cover, such that the external surfaces entering the sterile shroud remain sterile even if the underlying structure of the components entering the sterile shroud are non-sterile. For example, a portion of a non-sterile component may be covered by the external sterile cover to provide an exterior surface of the component that is sterile. After the non-sterile component is covered, the covered portion of the non-sterile component may be received in the sterile shroud, thereby maintaining sterility within the sterile shroud. The covered portion of the non-sterile component may then remain sterile while moving between the sterile field and the non-sterile field (i.e., by moving within the sterile shroud in the non-sterile field).

As shown in the embodiment of FIG. 1B, the shroud 124 is coupled to the support base 120 by a support member 122. The support base 120 may be mounted to or near a surgical table as described above with respect to manipulator assembly 102. The support base 120 may be, for example, a movable cart (e.g., a patient-side cart), a kinematic arm, a clamp, a wall-mounted manipulator, a ceiling-mounted manipulator, a table-mounted manipulator, or any other suitable mechanical support mechanism. In some embodiments, the support base 120 and the support member 122 are located in the non-sterile field for the duration of a medical procedure. The support member 122 is used to move and hold the position and/or orientation of the shroud 124 during the medical procedure. This movement can occur when the manipulator assembly 126 and its components are received within the shroud 124, partially received within the shroud 124, or outside of the shroud 124.

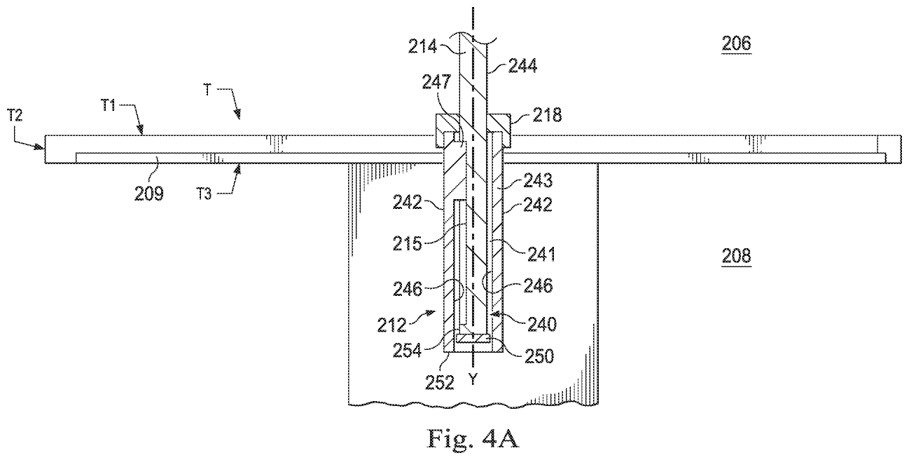

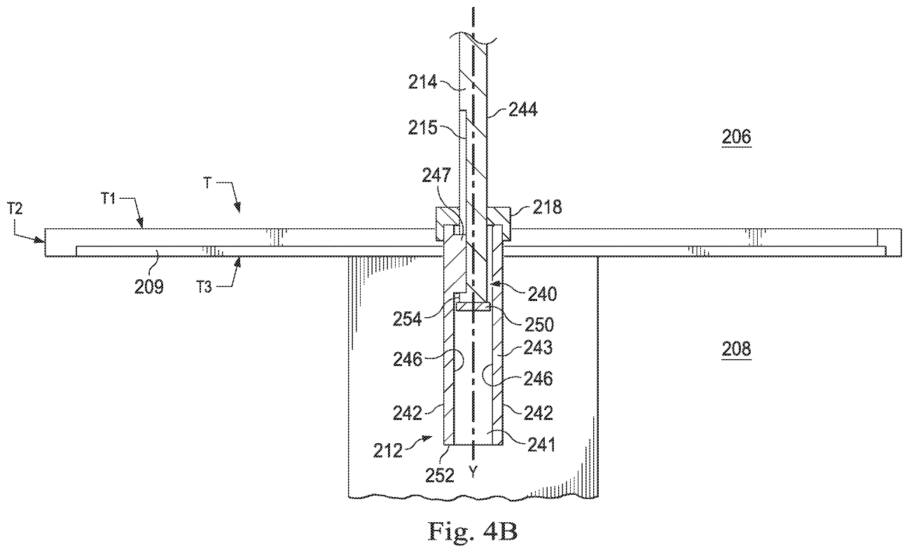

As will be discussed in greater detail below with respect to FIGS. 4A and 4B, the shroud 124 provides a sterile volume within the non-sterile field 154, which enables the portion of manipulator assembly 126 within the shroud 124 to remain sterile while moving between the sterile field and the non-sterile field. In addition, the shroud 124 enables a portion of a non-sterile component covered by an external sterile cover to remain sterile while moving between the sterile field and the non-sterile field.

FIG. 1C is similar to FIG. 1B, except the manipulator assembly 126 is coupled to a shroud 156, which is a variation of the shroud 124. In some embodiments, the shroud 156 is a re-shapeable member including both rigid and non-rigid sections. For example, the shroud 156 may be a bellows-like concave shape, an accordion-like concave shape, or any other suitable concave and/or elongated shape. The shroud 156 extends or contracts as needed during the medical procedure, extending as necessary to receive some or all of the manipulator assembly 126 as described above, and then contracting as some or all of the manipulator assembly 126 is withdrawn. The extension and contraction may be linear, non-linear, or a combination of linear and non-linear as needed during the surgical procedure. And, the components of the manipulator assembly 126 are received within the shroud 156 as described above with respect to the shroud 124. The ability of the shroud 156 to expand and contract as needed provides more space for clinical personnel to move near the operating table (e.g., at approximately thigh height) when some or all of the components of the manipulator assembly 126 are withdrawn from the shroud 156. This additional space is advantageous when, for example, changing an instrument mounted on a manipulator, operating a manual surgical instrument, or otherwise performing an action that requires direct patient access.

FIG. 2 is a perspective view of a patient coordinate space 200 in which teleoperated surgical manipulator assemblies 202, 204 are mounted on a side of a surgical table T according to some embodiments. In some embodiments, the manipulator assemblies 202, 204 are represented by manipulator assembly 102 in a medical procedure performed with system 100 and controlled by the control system 110. In other embodiments, the manipulator assemblies 202, 204 are represented by the manipulator assembly 126. In some examples, the manipulator assemblies 202, 204 may be used for non-teleoperational exploratory procedures or in procedures involving traditional manually operated surgical instruments, such as endoscopy. While only two manipulator assemblies 202, 204 are depicted, it is to be understood that more than two (e.g., three, four, five, six, and more than six) or fewer than two (i.e., one) manipulator assemblies can be included in some configurations.

As shown, the patient coordinate space 200 includes a sterile field 206 and a non-sterile field 208. These fields are typically separated by one or more boundaries that are conveniently identified and defined with reference to operating room equipment, such as the operating table T. The operating table T includes a top surface T1 (e.g., a surface on which the patient P is located), multiple side surfaces T2, and a bottom surface T3. An equipment rail 209 is attached to the table T along one of the side surfaces. In some embodiments, the sterile field 206 includes a portion of the patient coordinate space 200 that is above the operating table T. For example, a lower boundary of the sterile field 206 may be a horizontal plane that is coincident with, parallel with, or substantially parallel with the top surface T1 of the operating table T. In other examples, the lower boundary of the sterile field 206 may be a horizontal plane that is coincident with, parallel with or substantially parallel with a bottom surface T3 of the operating table T, a top surface of the rail 209, a bottom surface of the rail 209, or any other suitable plane as dictated by the needs of a particular surgical procedure. In some embodiments, an upper boundary of the sterile field 206 is a ceiling of the patient coordinate space 200 (e.g., the operating room). In various other embodiments, the sterile field 206 is defined by other boundaries, including non-horizontal boundaries. Persons familiar with surgery will understand that the table surface may be moved during surgery and the surface of the table may be angulated with respect to the plane of the floor, and so one or more sterile field boundaries may change dynamically during the operative procedure as the table surface moves. And, the table components provide a convenient reference to define a sterile field, although other physical references may be used such as the planes formed by the top surfaces of adjacent sterile tables and work surfaces.

In some embodiments, the non-sterile field 208 includes a portion of the space 200 that is below the top surface T1 of the operating table T. For example, an upper boundary of the non-sterile field 208 may be a horizontal plane that is coincident with, parallel with, or substantially parallel with the top surface T1 of the operating table T. In other examples, the upper boundary of the non-sterile field 208 may be a horizontal plane that is coincident with, parallel with, or substantially parallel with a bottom surface T3 of the operating table T, a horizontal plane between the top and bottom surfaces T1, T3 of the operating table T, a top surface of the rail 209, a bottom surface of the rail 209, or any other suitable horizontal plane. In some embodiments, a lower boundary of the non-sterile field 208 is a floor of the patient coordinate space 200 (e.g., the operating room). In various other embodiments, the non-sterile field 208 may be defined by other boundaries, including non-horizontal boundaries.

In alternative embodiments, the boundaries of the sterile field 206 and the non-sterile field 208 are defined with respect to a side table that may be present in the operating room and holds components of the manipulator assembly 202, for example, until the components are needed in the medical procedure. In other examples, the side table may hold one or more additional manipulator assemblies (e.g., manipulator assembly 204). The boundaries of the sterile field 206 and the non-sterile field 208 may also be defined with respect to any other structure in the operating room that holds, carries, touches, and/or transports sterile objects for use in the medical procedure.

In some embodiments, the table T may be moved or reconfigured during the surgery. For example, in some embodiments, the table T may be angulated or tilted about various axes, raised, lowered, pivoted, rotated, and the like. In some cases, such movements of the table T are integrated as a part of the teleoperated surgical manipulator system that includes the teleoperated surgical manipulator assemblies 202 and 204 and are controlled by the system. In alternative embodiments, different sections of the table T articulate independently of the other sections. For example, a top portion T4 of the table T may be tilted about various axes while a bottom portion T5 of the table T remains in an un-tilted position. In other examples, the bottom portion T5 of the table T is tilted about an axis while the top portion T4 of the table T is un-tilted. The table T includes two, three, four, or any other suitable number of independently articulable sections. Therefore, the boundaries of the sterile field 206 and the non-sterile field 208 are defined by one or more planar segments corresponding to one or more respective articulable sections of the table T.

The manipulator assembly 202 may be operated to move an instrument 211 within the space 200, and the manipulator assembly 204 may be operated to move an instrument 213 within the space 200.

The manipulator assembly 202 includes a manipulator 210 and a support structure 216. The manipulator 210 may include one or more drive systems, instrument interfaces, sterile adapters, or any other suitable component. The support structure 216 includes one or more links that support the manipulator 210 in space, such as the link 214. The support structure 216 is substantially similar to the component 128 in FIG. 1B. The manipulator assembly 204 includes a manipulator 220 and a support structure 226. The manipulator 220 may include one or more drive systems, instrument interfaces, sterile adapters, or any other suitable component. The support structure 226 includes one or more links that support the manipulator 220 in space, such as the link 224. The support structure 226 is substantially similar to the component 128 in FIG. 1B. The instrument 211 is coupled to the manipulator 210, and the instrument 213 is coupled to the manipulator 220. In some embodiments, the manipulators 210, 220 are, for example, standalone units including a system of drive mechanisms (not shown, e.g., motors).

In some embodiments, the manipulator 210 may be operated to move the instrument 211 as a whole in one or more degrees of freedom (DOFs). In addition, the manipulator 210 may optionally be operated to move one or more components of the instrument 211 in one or more DOFs. In several examples, the manipulator 210 may include at least one jointed pair or links (i.e., a kinematic pair), and the joint may be motorized. In some embodiments, some, but not all, kinematic pairs in a manipulator (e.g., the manipulator 210) may be motorized. An operator (e.g., a surgeon) controls the manipulator 210 to perform surgery. The manipulator 220 is similarly configured for operation of the instrument 213.

The manipulator 210 is movably coupled to the support structure 216, which includes the link 214, and the manipulator 220 is movably coupled to the support structure 226, which includes the link 224. In some embodiments, the support structure 216 may include one or more links that support the manipulator 210 in space. The links are typically movable so that the manipulator 210 may be placed at various positions in space. A joint between two links in the support structure 216 may be motorized or non-motorized. In some examples, the support structure 216 includes one or more motors, which may be teleoperated. However, the motor(s) of the support structure 216 is generally not operated as an operator moves an input device (e.g., one or more input devices of the user control system 106) to perform surgery. In some embodiments, a joint of the support structure 216 may be controlled by the operator, or a joint of the support structure 216 may be controlled by another suitable person, such as an operating room technician. The support structure 226 is similarly configured for support of the manipulator 220.

Any one or more of the components of the manipulator 210 and/or the manipulator 220 may be teleoperated. Thus, at least the manipulator 210 and/or the manipulator 220 are teleoperated components. During a medical procedure, the instruments 211, 213; the manipulators 210, 220; and the support structures 216, 226 operate within the sterile field 206. These components are sterilized prior to use in the medical procedure and maintain sterility during the medical procedure to prevent contamination of the sterile field 206. Alternately, one or more of the instruments 211, 213; the manipulators 210, 220; and/or the support structures 216, 226 may be covered in their own sterile drapes, sleeves, or coverings to ensure that their external surfaces are sterile even though the underlying structure of these components may not be sterile.

The manipulator assembly 202 is coupled to the table T by a coupling member 218 and a clamp 230. In some embodiments, the coupling member 218 is a joint (e.g., a ball joint, a spherical ball joint, a prismatic joint, a gimbal, and the like). The manipulator assembly 204 is coupled to the table T by a coupling member 228 and a clamp 233. The shroud 212 is coupled to the coupling member 218, and the shroud 222 is coupled to the coupling member 228. Each shroud 212, 222 is substantially similar to the shroud 124 in FIG. 1B. As will be described in greater detail below, the sterile link 214 is slidingly received within the shroud 212, and the sterile link 224 is slidingly received within the shroud 222 to preserve the sterility of each link. In some embodiments, the links 214, 224 may be non-sterile and covered by external sterile covers. In such embodiments, the non-sterile links 214, 224 and their respective external sterile covers may be slidingly received within the shrouds 212, 222 to preserve the sterility of the external sterile covers.

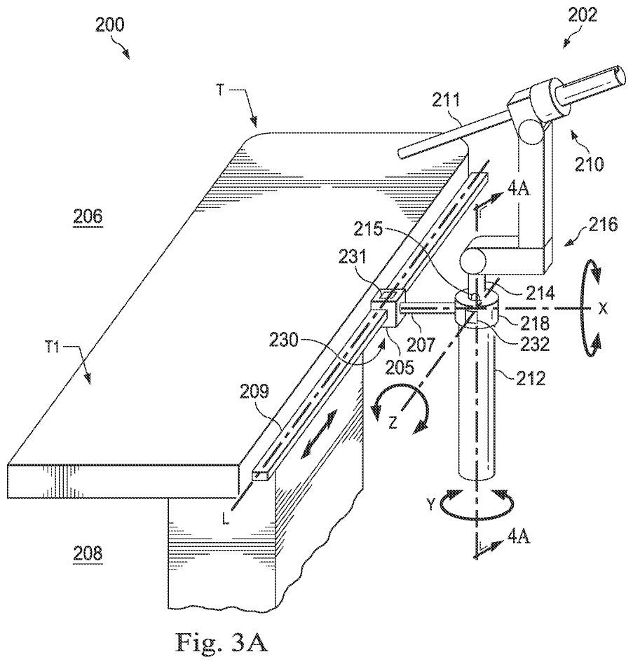

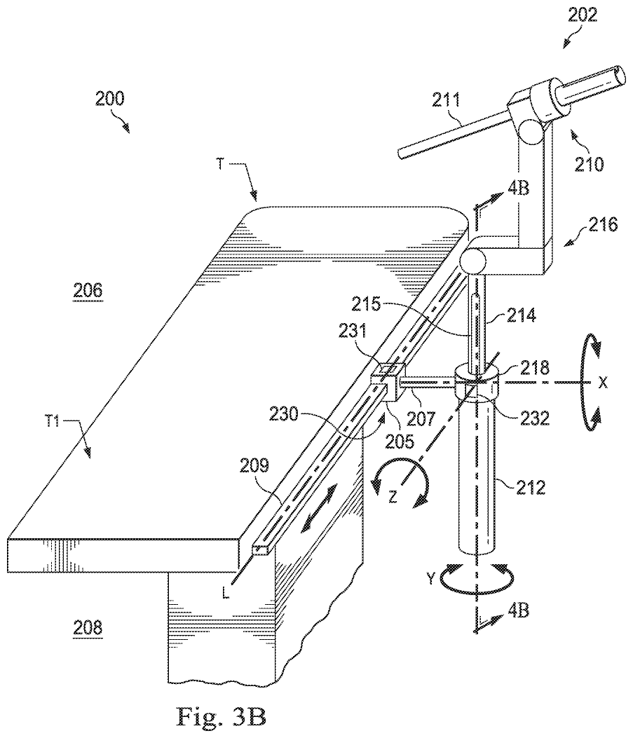

FIG. 3A illustrates an embodiment of the manipulator assembly 202 in greater detail. In some embodiments, the manipulator assembly 202 is coupled to the rail 209 of the table T by the clamp 230 (which may be an operating table clamp). The clamp includes a clamp body 205 and a link 207. The clamp body 205 may translate along the rail 209 to allow the position of the manipulator assembly 202 to be moved relative to the table T and the patient P. In some embodiments, the clamp 230 includes a lock 231 that is engaged to fixedly couple the manipulator assembly 202 to the rail 209 and prevent translation along the rail 209. Alternatively, the lock 231 may be positioned between the clamp 230 and the rail 209, in a coupling member 218, or in any other suitable location. In some examples, the operator O or an assistant can manually engage the lock 231. The lock 231 is engaged and/or disengaged as needed throughout the surgical procedure. In alternative embodiments, the clamp 230 may couple directly to the operating table T, to a support base of the operating table T, or to any other suitable component of the operating table T.

In the embodiment of FIG. 3A, the manipulator assembly 202 is coupled to the clamp 230 by the coupling member 218. In some embodiments, the coupling member 218 is a joint (e.g., a ball joint, a spherical ball joint, a prismatic joint, a gimbal, and the like). In embodiments where the coupling member 218 is a gimbal, the gimbal can include one or more degrees of freedom (DOFs), which may or may not intersect. The shroud 212 is coupled to the coupling member 218. The link 214 is supported by the coupling member 218 and extends through the coupling member 218 and into the shroud 212. The coupling member 218 thus provides a translational DOF for the shroud 212, relative to the table T. In some embodiments, the coupling member 218 includes three rotational DOFs, which allows the shroud 212 to rotate about one or more axes of the coupling member 218. For example, the shroud 212 can move in pitch, roll, and/or yaw DOF's about axes of the coupling member 218. In some cases, the link 214 can rotate independently within the shroud 212. In other cases, the shroud 212 and the link 214 are keyed to allow relative translational movement and prevent relative rotational motion (which will be discussed in further detail below with respect to FIG. 4A). In some embodiments, the link 207 and the shroud 212 or the link 207 and the coupling member 218 form a kinematic pair with a coupling member. The kinematic pair may have one or more degrees of freedom that allow the shroud 212 to rotate about as many degrees of freedom as are needed to provide the manipulator 210 with enough range of motion to place and orient the instrument 211 in a position that allows the instrument 211 to be inserted through a designated entry point in the patient P. In some examples, the shroud 212 rotates about the X or Z-axes with respect to the link 207.

As shown in FIG. 3B, the shroud 212 is movable in multiple degrees of freedom, which may include translational degrees of freedom. For example, the shroud 212 may be linearly translated along axis L. The shroud 212 may also be rotated about the X, Y, and Z-axes via the coupling member 218. The shroud 212 may also be translated along the Y-axis. The range of rotation may be limited by interference with the coupling member 218. For example, when pivoting about the X-axis and the Z-axis, the shroud 212 and the link 214 may encounter a sidewall of the coupling member 218, thus limiting the pivoting motion to less than 180°. In alternative embodiments, the translational and rotational degrees of freedom of the coupling member 218 may be distributed among multiple joints having one or more degrees of freedom.

In some embodiments, a locking mechanism 232 locks the coupling member 218 to prevent the shroud 212 and the link 214 from rotating about one or more of the X, Y, or Z-axes. In various embodiments, the locking mechanism 232 may be coupled to the clamp 230, the shroud 212, and/or the link 214. In other embodiments, the locking mechanism 232 is a component of the coupling member 218 itself. The operator O or an assistant may manually engage the locking mechanism 232 to lock the rotational degrees of freedom of the coupling member 218.

The link 214 is slidingly received within the shroud 212 and may be translated along the Y-axis relative to the shroud 212. In some examples, the link 214 may be slidingly received within a proximal portion of the shroud 212. As shown in FIG. 3A, the link 214 is retracted into the shroud 212, and as shown in FIG. 3B, the link 214 is extended from the shroud 212. In some embodiments, the translation of the link 214 relative to the shroud 212 may be performed manually or may be remotely manipulated in response to commands from the control system. In the embodiment of FIG. 3A, with the link 214 retracted within the shroud 212, the link 214 is contained within a sterile volume 240 of an inner channel 241 bounded by an inner surface 246 of a wall 243 of the shroud 212 (see FIG. 4A). During a procedure with the shroud 212 secured, the shroud 212 provides a static extension of the sterile field 206 for non-permanent ingress of the link 214. In some embodiments, the shroud 212 is a rigid cylindrical tube. In other embodiments, the shroud 212 may be another type of rigid structure such as a rectangular prism, a pentagonal prism, a hexagonal prism, or any other suitable elongated and/or concave shape. In still further embodiments, the shroud 212 is a non-rigid shroud (e.g., the shroud 212 is made of cloth, paper, a flexible polymer, rubber, a treated material, a laminated material, a layered material, or any other suitable flexible material.). In some embodiments, the link 214 is a cylindrical member that may be a tube or a solid shaft with a wall 244. In other examples, the link 214 may have a rectangular, pentagonal, hexagonal, or any other suitable cross-sectional shape. The link 214 may have a cross-sectional shape substantially the same as a cross-sectional shape of the shroud's inner channel 241. In some embodiments, an outer diameter of the link 214 is slightly smaller than a diameter of the inner channel 241 of the shroud 212 to allow a close fit.

As shown in FIG. 3B, the link 214 may include a groove 215 in the wall 244. An elongated protrusion 247 extends from the inner surface 246 of the shroud 212 to engage with the groove 215. The elongated protrusion 247 translates within the groove 215 when the link 214 extends and/or retracts within the shroud 212. The coupling of the groove 215 to the corresponding elongated protrusion 247 prevents the link 214 from rotating independently of the shroud 212. In other words, the groove 215 and the elongated protrusion 247 couple the rotational motion of the link 214 and the shroud 212 such that a commanded or passive rotation of either the link 214 or the shroud 212 about the Y-axis causes the coupled component to likewise rotate about the Y-axis.

While the embodiment of FIG. 3B shows one groove 215 in the wall 244 of the link 214, it is to be understood that more than one groove (e.g., two grooves, three grooves, or more than three grooves) can be included in the wall 244 of the link 214. In some embodiments, the grooves may be equidistantly spaced around the wall 244 of the link 214. For example, if there are two grooves, they may be spaced 180 degrees apart around the circumference of the wall 244 of the link 214. If there are three grooves, they may each be spaced 120 degrees apart around the circumference of the wall 244 of the link 214. If there are more than three grooves, they may similarly be equidistantly spaced around the circumference of the wall 244 of the link 214. Likewise, there may be more than one protrusion from the inner surface 246 of the link 214 to correspond with the same number of grooves.

In alternative embodiments, the groove 215 extends along substantially the entire length of the sterile link 214. This reduces the overall weight of the link 214 and, consequently, of the manipulator assembly 202. A reduction in weight allows for more efficient operation of the manipulator assembly 202 during a surgical procedure. A reduction in weight also reduces the load on certain components and/or joints of the manipulator assembly 202 which can reduce the amount of repairs needed and can lengthen the lifespan of the manipulator assembly 202. In other embodiments, the groove 215 extends along a portion that is less than substantially the entire length of the link 214 (e.g., two-thirds of the length, one-half of the length, one-third of the length, or any other length that is less than substantially all of the entire length of the link 214). This may reduce manufacturing costs because less machining and less time may be required to form the groove 215 in the outer wall of the link 214. In some alternative embodiments, the projections and grooves may be omitted with the link 214 permitted to rotate about the Y-axis within the shroud 212.

In some examples, the shroud 212 is a straight tube. For example, the outer surface 242 of the shroud 212 may be substantially perpendicular to a top surface T1 of the operating table T. Therefore, in some embodiments, the sterile link 214 may retract into the shroud 212 along a straight path. In other examples, the shroud 212 may have a curved tube. In such examples, the sterile link 214 may be a correspondingly curved solid or tubular member with the same or substantially similar curvature as the shroud 212. Therefore, in some embodiments, the sterile link 214 may retract into the shroud 212 along a curved path. In alternative embodiments, the shroud 212 is neither straight nor curved, such as, in a non-limiting example, when the shroud 212 is not rigid and is made of a flexible material. In such embodiments, the sterile link 214 may retract into the shroud 212 along an arbitrary, undefined path.

In some embodiments, the shroud 212 is reusable and able to withstand processing in an autoclave to become re-sterilized after each procedure.

FIG. 4A is a cross-sectional view of the link 214 and the shroud 212 in a retracted configuration along section line 4A-4A in FIG. 3A. FIG. 4B is a cross-sectional view of the link 214 and the shroud 212 in an extended position along section line 4B-4B in FIG. 3B. As shown in FIGS. 4A and 4B, a portion of the shroud 212 is positioned within the non-sterile field 208. As previously described, the inner surface 246 of the shroud 212 bounds the inner channel 241 that defines the sterile volume 240. Therefore, a portion of the sterile volume 240 extends within the non-sterile field 208 but remains sterile because of the shroud 212. Because the sterile volume 240 is defined by the inner surface 246 of the shroud 212, the sterile volume 240 translates and/or rotates with the shroud 212. More specifically, as the shroud 212 translates along the rail 209 of the table T and/or rotates about one or more axes X, Y, Z of the coupling member 218, the sterile volume 240 also translates along the rail 209 of the operating table T and/or rotates about the one or more axes of the coupling member 218. Therefore, a slidable, pivotable, and/or otherwise movable sterile volume 240 is positioned within and moveable within the non-sterile field 208. In examples when the shroud 212 is a tube, the shroud tube may include a solid side wall that defines the sterile volume 240.

The sterilized link 214 may be retracted within the shroud 212, as shown in FIG. 4A. When the link 214 is retracted within the shroud 212, a substantial portion of the link 214 is positioned within the shroud 212 and thus within the sterile volume 240. In such embodiments, the portion of the link 214 that is within the sterile volume 240 remains sterile. Thus, the portion of the link 214 within the sterile volume 240 is sterile even though that portion of the link 214 is also within the non-sterile field 208. The shroud 212 thus creates a protective sterility barrier that separates the non-sterile field 208 from the sterile volume 240. The portion of the link 214 that is not within the shroud 212 (and, therefore, not within the sterile volume 240) is also sterile because it is positioned within the sterile field 206. Therefore, in some embodiments, the entirety of the link 214 remains sterile before, during, and/or after the surgical procedure is performed, despite moving into and out of the non-sterile field 208.

In alternative embodiments, some or all of the components of the manipulator assembly 202 (e.g., the manipulator 210, the support structure 216, the instrument 211, and/or any other components of the manipulator assembly 202) may be positioned in or retracted within the shroud 212. Therefore, in some embodiments, the entirety of the manipulator assembly 202 remains sterile before, during, and/or after the surgical procedure is performed, despite traveling between the sterile field 206 and the non-sterile field 208. In addition, in embodiments where the manipulator assembly 202 includes non-sterile components that are covered by an external sterile cover, the external sterile cover remains sterile before, during, and/or after the surgical procedure is performed, despite traveling between the sterile field 206 and the non-sterile field 208. In various embodiments, the coupling member 218 defines an aperture from the sterile field to the region of the sterile field that has been extended into the non-sterile field by the sterile shroud. The coupling member may in some embodiments be a structural element that can support the aperture at the interface between the sterile and non-sterile fields in order to allow the structure of the manipulator to slidingly move in and out of the sterile shroud during the movements required for the surgical procedure.

As shown in FIGS. 4A and 4B, optionally in some embodiments, an end 252 of the shroud 212 may be open to the non-sterile field 208. The open end 252 may facilitate easier cleaning of the shroud 212. In addition, the open end 252 may help avoid entrapping air inside the shroud 212. FIGS. 4A and 4B additionally illustrate a collet 250 coupled to an end portion 254 of the sterile link 214. The collet 250 creates a seal between the sterile volume 240 and the non-sterile field 208. In some examples, the collet 250 creates a hermetic seal. In other examples, the collet 250 creates a seal that may allow air from the non-sterile field 208 to enter the sterile volume 240 but prevents an object such as a surgical apparatus, a surgical instrument, a cable, a drape, a cart, a human leg (e.g., the operator's leg), a human finger (e.g., the operator's finger), and/or any other object within the operating room from entering the sterile volume 240. In some embodiments, the collet 250 and the shroud 212 cooperate to prevent any objects from accidentally bumping into, contacting, or otherwise interfering with the sterile link 214. This may help maintain sterility of the sterile link 214 before, during, and/or after the surgical procedure is performed. Alternately, a distal portion of the shroud 212 may be closed. In such embodiments, the sterile link 214 includes an external sterile surface. In some embodiments, the link 214 is non-sterile and is covered by an external sterile cover, and the collet 250 and the shroud 212 cooperate to help maintain sterility of the external sterile cover.

The sterilized link 214 (or the non-sterile covered link) may be extended from the shroud 212 to a location outside of the shroud 212, as shown in FIG. 4B. When the link 214 is extended from the shroud 212, a substantial portion of the link 214 is positioned within the sterile field 206 with an end portion 254 of the sterile link 214 remaining within the sterile volume 240 defined by the inner surface 246 of the shroud 212. As shown in the embodiment of FIG. 4B, an overall size of the sterile volume 240 decreases as the link 214 is extended from the shroud 212. For example, as the link 214 extends from the shroud 212, the collet 250 translates along the Y-axis. As the collet 250 translates along the Y-axis toward the sterile field 206, the overall size of the sterile volume 240 decreases. As the link 214 retracts into the shroud 212 (as shown in FIG. 4A), the collet 250 translates along the Y-axis toward the non-sterile field 208, and the overall size of the sterile volume 240 increases.

During all of the extension, retraction, and rotation of the link 214 and the shroud 212, the entirety of the link 214 (or external sterile cover in some embodiments) remains in the sterile volume 240 and/or the sterile field 206. Therefore, the entirety of the sterile link 214 (or external sterile cover in some embodiments) remains sterile before, during, and/or after the surgical procedure is performed. In various alternative embodiments, the entire length of the shroud 212 may be positioned in the non-sterile field 208. Alternatively, a portion of the shroud 212 may extend, for example, above the plane of the top surface T1 of the table T and into the sterile field 206.

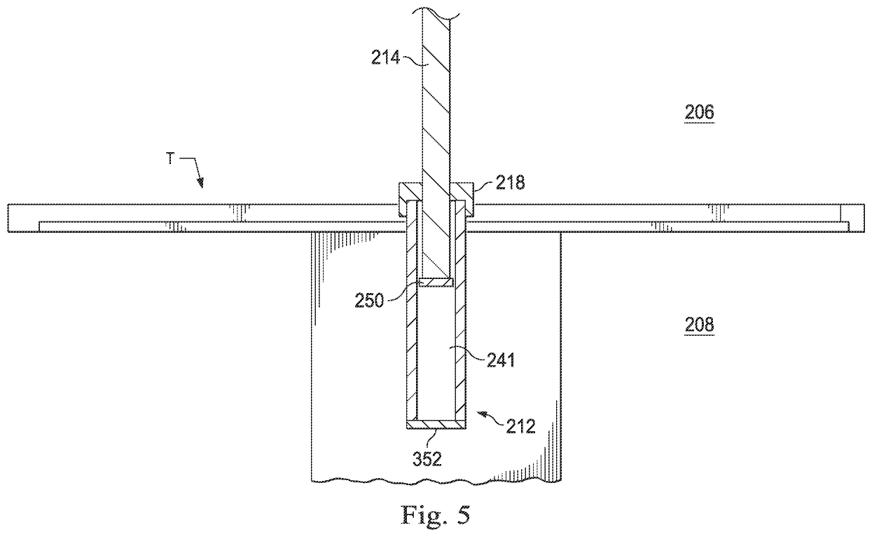

FIG. 5 is a cross-sectional view of the link 214 and the shroud 212 in an extended configuration where the shroud 212 includes an optional end cap 352. The end cap 352 may be placed on the end 252 of the shroud 212 to create a further barrier between the sterile volume 240 and the non-sterile field 208. The end cap 352 may extend into the inner channel 241 or may fit around the outside of the shroud 212 (e.g., around the outer surface 242 of the shroud 212). The end cap 352 may be coupled to the shroud 212 in any number of ways, such as a press fit, a snap fit, an adhesive connection, a welded connection, a threaded connection, or any other suitable coupling.

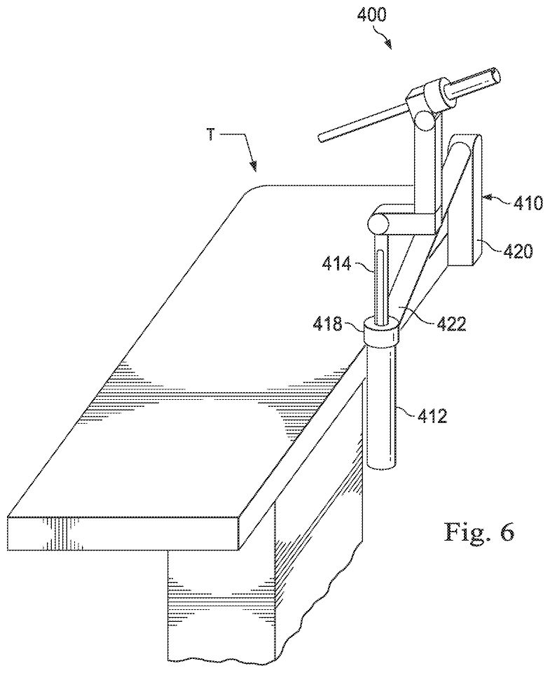

FIG. 6 is a perspective view of a manipulator assembly 400 coupled to a kinematic arm 410 mounted on a side of the table T according to some embodiments. The manipulator assembly 400 is substantially similar to the manipulator assembly 202. A link 414 of the manipulator assembly 400 is substantially similar to the link 214, a coupling member 418 is substantially similar to the coupling member 218, and a shroud 412 is substantially similar to the shroud 212. The kinematic arm 410 is coupled at an end 420 to the table T. The coupling to the table T may be direct, via a rail (e.g., rail 209) on the table, via a clamp (e.g., clamp 230), or another type of suitable connection. The kinematic arm 410 is coupled at an end 422 to the coupling member 418. In alternative embodiments, the end 422 may be coupled directly to the shroud 412 or the link 414. In some embodiments, the kinematic arm 410 and the shroud 412 form a kinematic pair with a joint having one or more degrees of freedom that allow the shroud 412 to rotate about the X or Z-axes with respect to the coupling member 418. In other embodiments, the kinematic arm 410 and the coupling member 418 form a kinematic pair with a joint having one or more degrees of freedom that allow the shroud 412 to rotate about the X or Z-axes with respect to the coupling member 418.

In some embodiments, the kinematic arm 410 may be manually manipulated to adjust the position of the manipulator assembly 400. In other embodiments, the kinematic arm 410 may be remotely manipulated by teleoperational control. For example, movement of the kinematic arm 410 may be controlled by the control system 110 (see FIG. 1A). The operator O may manipulate the user control system 106 (see FIG. 1A), which then manipulates the kinematic arm 410 via the control system 110. The kinematic arm 410 moves the shroud 412 and, therefore, the manipulator assembly 400 in any manner that is required for the surgical procedure. For example, the kinematic arm 410 may ascend, descend, translate laterally, rotate, and/or move the manipulator assembly 400 in any other direction. While the kinematic arm 410 manipulates the shroud 412, the link 414 remains within a sterile volume (e.g., the sterile volume 240) and/or a sterile field (e.g., the sterile field 206). Therefore, the entirety of the link 414 remains sterile before, during, and/or after the surgical procedure is performed.

FIG. 7A is a perspective view of a manipulator assembly 500 coupled to a kinematic arm 510 mounted on a manipulator platform 520 according to some embodiments. In some embodiments, a manipulator platform 520 is a patient-side cart or other movable unit. As shown in the embodiment of FIG. 7A, the manipulator platform 520 has wheels 522. Therefore, in some embodiments, the manipulator platform 520 may be moved around the surgical environment as needed before, during, and/or after the surgical procedure is performed in order to position the manipulator assembly 500 in a desired location. In various other examples, the manipulator platform 520 does not include wheels. In various embodiments, the manipulator platform 520 may be mounted near or adjacent an operating or surgical table T, or the manipulator platform 520 may be mounted or docked to the table T, or to a rail coupled to the table T, or integrally part of the table structure. The manipulator assembly 500 is substantially similar to the manipulator assembly 202. A link 514 of the manipulator assembly 500 is substantially similar to the link 214, a coupling member 518 is substantially similar to the coupling member 218, and a shroud 512 is substantially similar to the shroud 212. The kinematic arm 510 is coupled at an end 532 to the manipulator platform 520. In some embodiments, the end 532 of the kinematic arm 510 is coupled to a handle 524 of the manipulator platform 520. The kinematic arm 510 is coupled at an end 534 to the coupling member 518. In alternative embodiments, the end 534 is coupled directly to the shroud 512 or the link 514. In some embodiments, the kinematic arm 510 and the shroud 512 form a kinematic pair with a joint having one or more degrees of freedom that allow the shroud 512 to rotate about the X or Z-axes with respect to the coupling member 518. In other embodiments, the kinematic arm 510 and the coupling member 518 form a kinematic pair with a joint having one or more degrees of freedom that allow the shroud 512 to rotate about the X or Z-axes with respect to the coupling member 518. In some embodiments, the manipulator assembly 500 may have one or more sterilized components and/or one or more non-sterile components. The non-sterile components may be covered by external sterile covers. For example, in some embodiments, the link 514 and the kinematic arm 510 may be non-sterile components that are covered by external sterile covers. In some embodiments, a plurality of manipulator assemblies 500 may be coupled to the manipulator platform 520. Each of the manipulator assemblies 500 may be supported by a separate kinematic arm 510 that is coupled to the manipulator platform 520, and each of the manipulator assemblies 500 may be coupled to a separate shroud 512. In some embodiments, a common kinematic arm 510 may be used to support multiple manipulator assemblies 500.

In some embodiments, the kinematic arm 510 may be manually manipulated to adjust the position of the manipulator assembly 500. In other embodiments, the kinematic arm 510 may be remotely manipulated by teleoperational control. For example, movement of the kinematic arm 510 may be controlled by the control system 110 (see FIG. 1A). The operator O may manipulate the user control system 106 (see FIG. 1A), which then manipulates the kinematic arm 510 via the control system 110. The kinematic arm 510 may move the shroud 512 and, therefore, the manipulator assembly 500 in any manner that is required for the surgical procedure. For example, the kinematic arm 510 may ascend, descend, translate laterally, rotate, and/or move the manipulator assembly 500 in any other direction. While the kinematic arm 510 manipulates the shroud 512, the link 514 remains within a sterile volume (e.g., the sterile volume 240) and/or a sterile field (e.g., the sterile field 206). Therefore, the entirety of the link 514 remains sterile before, during, and/or after the surgical procedure is performed. In some embodiments, the shroud 512 may be positioned inside the manipulator platform 520. For example, in some embodiments, the manipulator platform 520 may include an internal chamber that at least partially houses the shroud 512. In some embodiments, the internal chamber of the manipulator platform 520 may complete house the shroud 512. In embodiments having a plurality of manipulator assemblies 500 coupled to the manipulator platform 520, the internal chamber may house a plurality of shrouds 512 and/or the manipulator platform 520 may have a plurality of chambers to separate house the shrouds 512.

In alternative embodiments, the end 532 of the kinematic arm 510 may be coupled to a ceiling-mounted manipulator, a wall-mounted manipulator, or to another component in the surgical environment.

FIG. 7B is a perspective view of a manipulator assembly 500 coupled to a kinematic arm 510 mounted on a manipulator platform 520 according to some embodiments. In the embodiment of FIG. 7B, the manipulator assembly 500 is covered by an external sterile cover 700 and the manipulator assembly 500 may be non-sterile. In various embodiments, the external sterile cover 700 may be a sterile drape, sterile sleeve, or sterile cover. The external sterile cover 700 is received in a sterile shroud 720 extending from a portion of the manipulator platform 520. The sterile shroud 720 defines a sterile volume in the shroud 720. An actuated portion 730 of the manipulator assembly 500 can slidably translate into and out of the sterile shroud 720 while maintaining sterility of the external sterile cover 700. The sterile shroud 720 may be held in place by an aperture 710, which may be part of the kinematic arm 510. In some embodiments, the sterile shroud 720 may at least partially extend into the manipulator platform 520 (e.g., in an internal chamber of the manipulator platform 520). In some embodiments, the sterile shroud 720 may be formed by an inverted portion of the external sterile cover. For example, the external sterile cover 700 may be placed over the manipulator assembly 500 and then folded back (i.e., inverted) over a lower portion of the manipulator assembly 500 that has already been covered by the external sterile cover 700 to form the sterile shroud 720. A sterile volume may then be defined as the space inside of the inverted portion of the sterile shroud 720 (i.e. the volume accessible inside aperture 710 supported by kinematic arm 510). In an alternate embodiment, the aperture 710 can be attached directly to the operating table within the sterile field rather than be attached to kinematic arm 510 or aperture 710 can be attached to the operating table via kinematic arm 510 which can in turn be attached to a portion of the operating table in the non-sterile field rather than to the manipulator platform 520. In both of these alternate embodiments, the function of the sterile shroud 720 and aperture 710 remains the same as the embodiment where aperture 710 is attached to kinematic arm 510 which is attached to manipulator platform 520.

While the embodiments above are discussed in the context of medical or surgical procedures, it is to be understood that the systems, instruments, and methods may also be used for non-medical purposes. For example, the systems, instruments, and methods may be used for non-surgical diagnosis, industrial systems, general robotic systems, and general teleoperational systems.

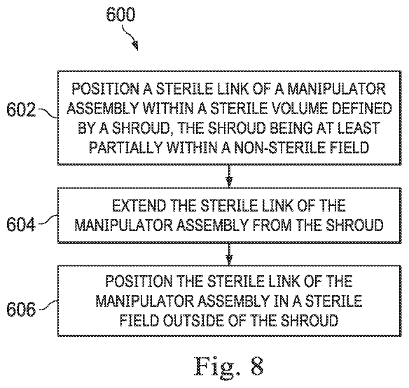

FIG. 8 illustrates a method 600 for extending a sterile link or a covered non-sterile link from a non-sterile field to a sterile field according to some embodiments. The method 600 is illustrated as a set of operations or processes 602 through 606 and is described with continuing reference to FIGS. 1A-7B. The processes 602 through 606 will be described with reference to a sterile link, however, in alternate embodiments, the link may be a non-sterile link that is covered by an external sterile cover. Not all of the illustrated processes 602 through 606 may be performed in all embodiments of method 600. Additionally, one or more processes that are not expressly illustrated in FIG. 6 may be included before, after, in between, or as part of the processes 602 through 606. In some embodiments, one or more of the processes 602 through 606 may be implemented, at least in part, in the form of executable code stored on non-transitory, tangible, machine-readable media that when run by one or more processors (e.g., the processors of a control system) may cause the one or more processors to perform one or more of the processes. In one or more embodiments, the processes 602 through 606 may be performed by the control system 110.

At a process 602, a sterile link (e.g., the link 214) of a manipulator assembly is positioned within a sterile volume defined by a shroud (e.g., the shroud 212). The shroud is located at least partially within a non-sterile field. The non-sterile field may be the non-sterile field 208. In some embodiments, the sterile link 214 may be fully retracted within the sterile volume 240. In other embodiments, the sterile link 214 may be substantially, but not fully, retracted within the sterile volume 240. In still other embodiments, all of the components of the support structure 216 may be substantially, but not fully, retracted within the sterile volume.

At a process 604, the sterile link of the manipulator assembly is extended from the shroud. In some embodiments, an operator may manually extend the sterile link 214 from the shroud 212, or the shroud 212 may be remotely manipulated in response to commands from the control system.

At a process 606, the sterile link is positioned in a sterile field outside of the shroud. For example, the sterile link 214 extended from the shroud 212 is positioned in the sterile field 206.

In some embodiments, the processes 602 through 606 may be reversed while maintaining sterility of the sterile link 214. For example, the sterile link 214 may initially be positioned in the sterile field outside of the shroud (process 606). The sterile link 214 may then be positioned within the sterile volume defined by the shroud (process 602), and the link 214 may be extended into the shroud.

In some embodiments, the method 600 may further include the process of moving the sterile volume 240 by moving the shroud 212 from a first position to a second position within the non-sterile field 208. In some embodiments, the method 600 may further include the process of locking the shroud 212 at the second position. In some embodiments, an operator may manually lock the shroud 212 in a desired position and/or orientation, such as a fully extended position. For example, a locking mechanism positioned in, on, or near the coupling member 218 may engage the shroud 212 and prevent the shroud 212 from moving and/or rotating. In other examples, the locking mechanism may engage the sterile link 214 and prevent the sterile link 214 from moving and/or rotating.

In some embodiments, the method 600 may further include the step of removably clamping the shroud 212 to an operating table. In some embodiments, the shroud 212 may be removably coupled to an operating table T via a clamp 230. In some embodiments, the shroud 212 may be removably coupled to the operating table T. In other embodiments, the method 600 may further include the step of locking the sterile link 214 of the manipulator assembly at the position outside of the shroud 212.

One or more elements in embodiments of this disclosure may be implemented in software to execute on a processor of a computer system such as a control processing system. When implemented in software, the elements of the embodiments of the present disclosure are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc.

Note that the processes and displays presented may not inherently be related to any particular computer or other apparatus, and various systems may be used with programs in accordance with the teachings herein. The required structure for a variety of the systems discussed above will appear as elements in the claims. In addition, the embodiments of the present disclosure are not described with reference to any particular programming language. It will be appreciated that a variety of programming languages may be used to implement the teachings of the present disclosure as described herein.

While certain exemplary embodiments of the present disclosure have been described and shown in the accompanying drawings, it is to be understood that such embodiments are merely illustrative of and not restrictive on the broad invention, and that the embodiments of the present disclosure not be limited to the specific constructions and arrangements shown and described, since various other modifications may occur to those ordinarily skilled in the art.

1. A system comprising:
a shroud that defines a sterile volume; and
a computer assisted manipulator assembly comprising a link slidable within the sterile volume, wherein the link includes an external sterile surface or is covered by an external sterile cover positioned at least partially between the shroud and the link.

2. (canceled)

3. The system of claim 1, wherein the link is non-sterile and is covered by the external sterile cover, the external sterile cover being a sterile drape, sterile sleeve, or sterile cover.

4. The system of claim 3, wherein the shroud is formed by an inverted portion of the external sterile cover.

5. The system of claim 1, wherein at least a portion of the sterile volume created by the shroud extends into a non-sterile field in a surgical environment.

6. The system of claim 5, wherein the non-sterile field is defined below a plane defined by a top surface of an operating table.

7-10. (canceled)

11. The system of claim 1 further comprising:
an operating table comprising a top surface, wherein:
a sterile field for surgery is defined above the top surface of the operating table, and a non-sterile field is defined below the top surface of the operating table;
the shroud is coupled to the operating table, and at least a portion of the sterile volume of the shroud extends into the non-sterile field; and
the link of the manipulator assembly extends from the sterile volume of the shroud into the sterile field.

12-14. (canceled)

15. The system of claim 1, wherein at least a portion of the shroud is straight such that the link of the manipulator assembly is slidingly received within the sterile volume along a straight path.

16. (canceled)

17. (canceled)

18. The system of claim 1, further comprising:
an operating table clamp,
wherein the shroud is coupled to the operating table clamp at a coupling member having one or more rotational degrees of freedom.

19-21. (canceled)

22. The system of claim 1, further comprising:
an operating table clamp,
wherein the shroud is coupled to the operating table clamp at a coupling member having one or more translational degrees of freedom separate from a degree of freedom defined by the link of the manipulator assembly sliding within the sterile volume of the shroud.

23. (canceled)

24. The system of claim 22, wherein:
at least a portion of the shroud extends into a non-sterile field; and
translation of the coupling member moves at least a portion of the sterile volume of the shroud within the non-sterile field.

25. The system of claim 1, wherein:
the link of the manipulator assembly is slidingly received within the sterile volume of the shroud at a proximal portion of the shroud; and
a distal portion of the shroud is closed.

26-33. (canceled)

34. A system comprising:
a shroud that defines a sterile volume, wherein the shroud is couplable to a table; and
a computer assisted manipulator assembly comprising a link slidingly received within the sterile volume, wherein the link includes an external sterile surface or is covered by an external sterile cover positioned at least partially between the shroud and the link,
wherein a sterile field includes a lower boundary defined by a first plane along a first surface of the table,
wherein a non-sterile field includes an upper boundary defined by a second plane extending below or coincident with the first plane, and
wherein at least a portion of the shroud is positioned in the non-sterile field such that a portion of the sterile volume extends below the second plane into the non-sterile field.

35. The system of claim 34, wherein at least a portion of the shroud is straight such that the link of the manipulator assembly is slidingly received within the sterile volume along a straight path.

36. The system of claim 34, wherein at least a portion of the shroud is curved such that the link of the manipulator assembly is slidingly received within the sterile volume along a curved path.

37. The system of claim 34, wherein the shroud comprises a tube comprising a solid side wall that defines the sterile volume of the shroud.

38. The system of claim 34, further comprising:
a table clamp,
wherein the shroud is coupled to the table clamp at a coupling member having one or more translational degrees of freedom separate from a degree of freedom defined by the link of the manipulator assembly sliding within the sterile volume of the shroud.

39. A system comprising:
a shroud that defines a sterile volume; and
a computer assisted manipulator assembly comprising a non-sterile elongate link slidable within the sterile volume, wherein the elongate link is covered by an external sterile cover that extends at least partially between the shroud and the elongate link as the elongate link slides within the sterile volume.

40. The system of claim 39, wherein the shroud is formed by an inverted portion of the external sterile cover.

41. The system of claim 39, wherein at least a portion of the shroud is straight such that the elongate link of the manipulator assembly is slidingly received within the sterile volume along a straight path.

42. The system of claim 39, further comprising an operating table clamp, wherein:
the shroud is coupled to the operating table clamp at a coupling member having one or more translational degrees of freedom separate from a degree of freedom defined by the elongate link of the manipulator assembly sliding within the sterile volume of the shroud;
at least a portion of the shroud extends into a non-sterile field; and
translation of the coupling member moves at least a portion of the sterile volume of the shroud within the non-sterile field.
